(19) United States
(12) Patent Application Publication
(10) Pub. No.: US 2002/0054911 A1
(43) Pub. Date: May 9, 2002

(54) Novel oral dosage form for carvedilol

(75) Inventor: Choon K. Oh, Chester Springs, PA (US)

Correspondence Address: GLAXOSMITHKLINE, Corporate Intellectual Property - UW2220, P.O. Box 1539, King of Prussia, PA, 19406-0939, US

(73) Assignee: Boehringer Mannheim Pharmaceutical Corporation-Sm ithKline Beckman Corporation, Limited Partnershi

(21) Appl. No.: 09/974,460

(22) Filed: Oct. 9, 2001

Related U.S. Application Data
(63) Continuation of application No. 09/554,307, filed on May 11, 2000.

(51) Int. Cl. A61K 9/14; A61K 31/403

(52) U.S. Cl. 424/488; 514/411

(57) ABSTRACT

The present invention discloses a matrix formulation containing carvedilol.

### FIELD OF THE INVENTION

The present invention relates to a novel formulation containing carvedilol, or a pharmaceutically acceptable salt thereof, and to its use in the treatment and/or prophylaxis of certain disorders.

### BACKGROUND OF THE INVENTION

U.S. Pat. No 4,503,067 describes a compound which is known as carvedilol. This compound is a novel multiple action drug useful in the treatment of mild to moderate hypertension. Carvedilol is known to be both a competitive non-selective β-adrenoceptor antagonist and a vasodilator. The vasodilatory actions of carvedilol result primarily from α₁-adrenoceptor blockade, whereas the β-adrenoceptor blocking activity of the drug prevents reflex tachycardia when used in the treatment of hypertension. These multiple actions of carvedilol are responsible for the antihypertensive efficacy of the drug. Also, carvedilol, as a consequence of its antioxidant action in attenuating oxygen free radical-initiated lipid peroxidation, is useful in organ protection, in particular, cardioprotection. Additionally, carvedilol is useful in the treatment of congestive heart failure.

The current formulation of carvedilol is a conventional swallow tablet, taken twice daily. This formulation is in immediate release form; that is to say the nature of the formulation is such that by the time carvedilol leaves the stomach, it is either in solution or it is in the form of a suspension of fine particles, i.e., a form from which carvedilol can be readily absorbed.

It has now been found that controlled release and delayed release formulations containing carvedilol give rise to a once daily formulation. These formulations are able to extend the duration of action of carvedilol, thus improving the bioavailability of this drug.

### SUMMARY OF THE INVENTION

The present invention provides a controlled release or delayed release formulation containing carvedilol or a pharmaceutically acceptable salt thereof.

### DETAILED DESCRIPTION OF THE INVENTION

The present invention provides a controlled release or delayed release formulation comprising carvedilol, which is (1-(carbazol-4-yloxy-3-[[2-(o-methoxyphenoxy)-ethyl]amino]-2-propanol), of the formula (I):

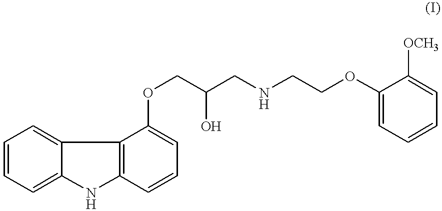

or a pharmaceutically acceptable salt thereof, in an oral dosage unit form.

The present invention also provides for a matrix formulation comprising carvedilol in an oral dosage unit form and for an enteric coated formulation comprising carvedilol in an oral dosage unit form.

Carvedilol may be conveniently prepared as described in U.S. Pat. No. 4,503,067. Reference should be made to said patent for its full disclosure, the entire disclosure of which is incorporated herein by reference.

According to the formulation of the instant invention, carvedilol is suitably in the form of the free base or a pharmaceutically acceptable salt thereof. Preferably, carvedilol is in the form of the free base.

By controlled release is meant any formulation that achieves slow release of drug over an extended period of time. In the controlled release formulations of the instant invention, a portion of the carvedilol in the formulation is made available as a priming dose and the remainder is released in a sustained fashion. An example of a controlled release system is a matrix formulation.

By delayed release is meant any formulation that utilizes repetitive, intermittent dosings of carvedilol from one or more immediate release units incorporated into a single dosage form. Examples of delayed release systems include repeat action tablets and capsules, and enteric-coated tablets where timed release is achieved by a barrier coating.

Examples of controlled release formulations which are suitable for incorporating carvedilol are described in:

Sustained Release Medications, Chemical Technology, Review No. 177, Ed. J. C. Johnson, Noyes Data Corporation (1980); and

Controlled Drug Delivery, Fundamentals and Applications, 2nd Edition, Eds. J. R. Robinson, V. H. L. Lee, Mercel Dekkes Inc., New York (1987).

Examples of delayed release formulations which are suitable for incorporating carvedilol are described in:

Remington's Pharmaceutical Sciences, 16th Edition, Ed. A. Osol, Mack Publishing Company (1980).

Other examples of controlled release formulations which are suitable for incorporating carvedilol are described in U.S. Pat. No. 4,839,177, issued Jun. 13, 1989, and U.S. Pat. No. 5,422,123, issued Jun. 6, 1995. Matrix controlled release formulations for carvedilol are detailed in U.S. Pat. No. 4,389,393, issued Jun. 21, 1983, and U.S. Pat. No. 4,968,508, issued Nov. 6, 1990.

Additionally, the controlled release formulations containing carvedilol may be in the form of a non-compressed pellet, having an enteric coat or a sustained release coat permeable to gastrointestinal juices. These controlled release formulations are prepared, for example, as described in U.S. Pat. No. 4,524,060, issued Jun. 18, 1985, and U.S. Pat. No. 4,983,401, issued Jan. 8, 1991. Other controlled release formulations are described in U.S. Pat. No. 4,880,830, issued Nov. 14, 1989, and U.S. Pat. No. 5,068,112, issued Nov. 26, 1991.

Such controlled release formulations are preferably formulated in a manner such that release of carvedilol is affected predominantly during the passage through the stomach and the small intestine, and delayed release formulations are preferably formulated such that release of the carvedilol is avoided in the stomach and is affected predominantly during passage through the small intestine

Said formulations are preferably formulated such that the release of the carvedilol is predominantly 1½ to 3 hours post ingestion.

The small intestine is suitably the duodenum, the ileum or the jejunem.

The formulations of the present invention allow for once-a-day dosing.

Preferred formulations for carvedilol are enteric coated tablets or caplets, wax or polymer coated tablets or caplets or time-release matrices, or combinations thereof. The oral route of administration of the formulation of the present invention is preferred.

According to the instant invention, the controlled release formulation may be a matrix formulation. This formulation may comprise a plurality of matrix cores containing carvedilol, said matrix cores having different release rates of the drug. The preferred formulation comprises an immediate release phase of carvedilol, as well as a sustained release phase. The sustained release phase matrix core may be uncoated or coated with a release-delaying substance. Preferably, when the matrix core is coated with a release-delaying substance, the release-delaying substance is present in an amount of from 2 to 30% (w/w) relative to the matrix core. More preferably, the release-delaying substance is present in an amount of from 5 to 25% (w/w).

The release-delaying substance of the present invention is a coating agent or a blend of agents thereof, which protects carvedilol from immediate degradation in the stomach. The overcoating, depending on the release rate desired, may allow for continual release, or slow release, or delayed release. A preferred release-delaying substance is enteric coating, i.e., a medicinal preparation treated to pass through the stomach unaltered, which disintegrates in the intestines.

The matrix formulations of the present invention may be prepared using three types of materials: insoluble plastics, hydrophilic polymers or fatty compounds. Plastic matrices include methyl acrylate-methacrylate, polyvinyl chloride and polyethylene. Hydrophilic polymers include methylcellulose, hydroxypropylmethylcellulose (HMPC) and sodium carboxymethylcellulose. Fatty compounds include various waxes such as carnauba wax and glyceryl tristearate. The most common method of preparation is to mix carvedilol with the matrix material and then compress the mixture into tablets. In the case of wax matrices, carvedilol is generally dispersed in molten wax, which is then congealed, granulated and compressed into cores. In the matrix formulation containing carvedilol, the priming dose (the portion of the carvedilol that is immediately available in the formulation) is placed in a coat of the tablet. The coat can be applied by press coating or by conventional pan or air suspension coating.

In one embodiment of the invention, the carvedilol matrix tablet formulation comprises a mixture of HMPC and Carbopol. In a further embodiment of the invention, the carvedilol matrix tablet formulation comprises a mixture of HMPC, Carbopol and mannitol. The flow diagram hereinafter summarizes the manufacturing process for the preparation of controlled release tablets containing carvedilol.

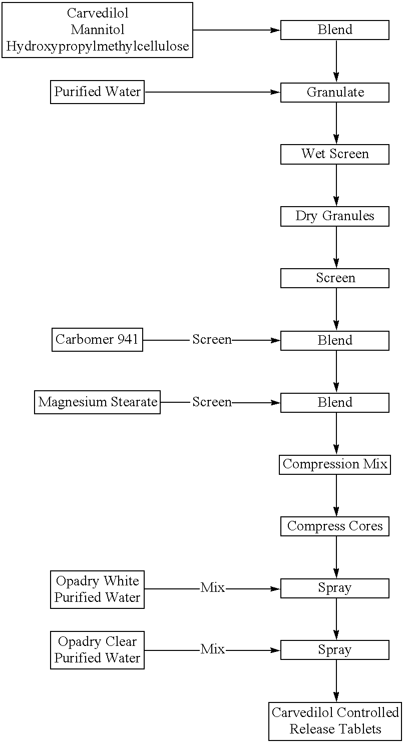

According to the instant invention, carvedilol, mannitol, and HPMC is granulated with purified water, wet screened, and then dried. The dry granules are screened. The resultant internal granulation is blended with pre-screened Carbomer 941 until homogeneous. Pre-screened magnesium stearate is mixed with the blend to create the compression mix. Tablets are compressed as round cores and are coated to an approximate 3% weight gain with an Opadry® white solution, followed by an approximate 0.5% weight gain with an Opadry® clear solution.

The present invention also provides for various combinations of immediate release and controlled release forms. For example, the uncoated sustained release matrix core may be in combination with an immediate release form of carvedilol and/or a coated matrix form. The matrix core may be comprised of a multitude of pellets coated independently with different release-delaying substances, all of which may be combined with uncoated or immediate release forms of carvedilol.

Delayed release formulations containing carvedilol may be prepared either by coating particles or granules of carvedilol with varying thicknesses of slowly soluble polymers, or by microencapsulation. In formulations employing microencapsulation, a hydrophilic substance acts as the coating material around a microcapsule. The hydrophilic substance can be selected from a variety of natural and synthetic polymers including shellacs, waxes, starches, cellulose acetate phthlate or butyrate, polyvinylpyrrolidone and polyvinyl chloride. Once the coating material dissolves, all the carvedilol in the microcapsule is immediately available for dissolution and absorption. Thus, the release of carvedilol can be controlled by adjusting the thickness and the dissolution rate of the coat. The thickness can be varied from less than 1 micromolar to 200 micromolar by changing the amount of coating material from 3 to 30% of the total weight. If only a few different thicknesses are used, usually three or four, carvedilol will be released at different predetermined times to give a delayed release effect, i.e., repeat action. If a spectrum of different thicknesses is employed, a more uniform blood level of carvedilol can be obtained. The coated particles can be directly compressed into tablet, or placed in capsules.

Carvedilol in the form of a controlled release or delayed release formulation can be used to treat hypertension, angina and congestive heart failure. The formulations of the instant invention may also be used in organ protection, for example, in cardioprotection.

The present invention provides a method of treating hypertension, angina and congestive heart failure by administering an effective amount of a controlled release or delayed release formulation containing carvedilol or a pharmaceutically acceptable salt thereof, to a sufferer in need thereof.

The present invention further provides the use of a controlled release or delayed release formulation containing carvedilol or a pharmaceutically acceptable salt thereof in the manufacture of a medicament, for treating hypertension, angina and congestive heart failure.

The present invention also provides a pharmaceutical composition for use in the treatment of hypertension, angina and congestive heart failure which comprises a controlled release or delayed release formulation, preferably a matrix formulation, containing carvedilol or a pharmaceutically acceptable salt thereof.

No unacceptable toxicological effects are expected when carvedilol is used according to the present invention.

The examples which follow are not intended to limit the scope of this invention, but are provided to illustrate this invention. Many other embodiments will be readily apparent to those skilled in the art.

### EXAMPLES

### Manufacturing Process Description

### Blending

Step 1. Weigh out the exact amounts of carvedilol, mannitol, hydroxypropyl methylcellulose, and purified water.

Step 2. Transfer the carvedilol, mannitol, and hydroxypropyl methylcellulose into a high shear mixer product bowl.

Step 3. Pre-blend ingredients for 2 minutes with the impeller and chopper at low speed setting.

### Granulation

Step 4. Granulate with purified water at low speed until desired granule appearance is achieved.

Step 5. Discharge granulation into stainless steel container for the wet-milling process.

Step 6. Slowly add the wet granules through the Quadro Comil (with screen) into a stainless steel container.

Step 7. Transfer the milled granulation to the pre-heated fluid bed product bowl.

Step 8. Dry the granules by maintaining the target inlet temperature of approximately 70° C. (65° C.-75° C.) until the product temperature reaches the target temperature (40-47° C.) and the loss on drying is within the target range (0.5-1.8%).

Step 9. Set-up the Quadro Comil (variable speed) and attach the screen for milling.

Step 10. Add the dry granules through the Quadro Comil (with screen) into pretared polyethylene bags.

### Unlubricated Granulation Mix

Step 11. Screen an excess amount of Carbomer 941 (Carbopol 971P) to deaggregate by passing though a #20 mesh stainless steel screen by hand.

Step 12. Weigh out the exact amount of pre-screened Carbomer 941 (Carbopol 971P) onto the weigh paper.

Step 13. Weigh out the exact amount of carvedilol internal granulation into properly labelled polyethylene bags.

Step 14. Set-up a suitable size V-Blender.

Step 15. Transfer ⅓rd of the carvedilol internal granulation into the ‘V’ blender.

Step 16. Add ⅓rd of the Carbomer 941 (Carbopol 971P) to the ‘V’ blender.

Step 17. Repeat Steps 15 and 16 until all internal granulation and Carbomer 941 (Carbopol 971P) is in the ‘V’ blender.

Step 18. Mix for 30 minutes or until homogeneous.

Step 19. Remove samples for in-process testing.

### Lubricated Granulation Mix

Step 20. Screen an excess amount of magnesium stearate (to de-aggregate) by passing though a #40 mesh stainless steel screen by hand.

Step 21. Weigh out the exact amount of pre-screened magnesium stearate onto the weigh paper.

Step 22. Load the magnesium stearate into the blender (containing the unlubricated granulation) and mix for 3 minutes. Compression

Step 23. Transfer the compression mix to the hopper of a rotary tablet press using 7/16″×⅝″ round standard tooling.

Step 24. Compress tablets to meet the physical properties targets.

Step 25. Remove samples for in-process testing throughout the run.

### Coating

Step 26. Separately weigh out the exact amount of carvedilol round active cores, Opadry® White and Opadry® Clear into polyethylene bags. If necessary, the round active cores may be bulked using oval placebo cores to achieve the batch size necessary to fill the coating pan.

Step 27. Transfer into a suitable, clean tared container, the required quantity of purified water to produce a 12% solids concentration of Opadry® White.

Step 28. With a vortex mixing action, slowly add the Opadry® White to the purified water. Continue mixing until no solid constituents are visible. Use this solution within 24 hours of manufacture.

Step 29. Transfer into a suitable, clean tared container, the required quantity of purified water to produce a 5% solids concentration of Opadry® Clear. e

Step 30. With a vortex mixing action, slowly add the Opadry® Clear to the Purified Water. Continue mixing until no solid constituents are visible. Use this solution within 24 hours of manufacture.

Step 31. Set-up the Accela Coater coating pan. Set pump to deliver white and clear coating solution to spray at a rate of approximately 35 g/minute.

Step 32. Transfer the cores to the coating pan. Pre heat the cores: Set the inlet temperature to 55° C. (40° C.-70° C.) while jogging the pan periodically. When product temperature reaches approximately 42° C. (37° C.-45° C.) start spray. Spray the entire quantity of white coating solution to obtain approximately a 3% weight gain coat. Follow with clear coating solution to obtain approximately a 0.5% weight gain coat.

Step 33. Remove coated tablets from coating pan into double polyethylene-lined drum. If placebo cores were used to bulk up the coating batch size, a sorting/inspection process is performed after completion of the coating run, to separate the oval placebo cores from the round active cores.

### Example 1

TABLE 1
Unit Formulae for Controlled Release Carvedilol Formulations

| Strength | | 50 mg | 50 mg | 50 mg |
|---|---|---|---|---|
| Formula | | BC | BD | BE |
| Component | Compendia | Quantity mg/tablet | | |
| Carvedilol | | 50.0 | 50.0 | 50.0 |
| Mannitol | USP | 152.5 | 366.25 | 360.0 |
| Hydroxypropyl Methy- | USP | 37.5 | 75.0 | 75.0 |
| cellulose | | | | |
| Carbomer 934P | NF | 7.5 | 3.75 | 10.0 |
| Magnesium Stearate | NF or Ph. Eur. | 2.5 | 5.0 | 5.0 |
| Opadry White | NC | 7.5 | 15.0 | 15.0 |
| (OY-S-9603) | | | | |
| Opadry Clear | NC | 1.25 | 2.5 | 2.5 |
| (YS-1-19025A) | | | | |
| Purified Water | USP or Ph. Eur. | q.s | q.s. | q.s. |
| Total Tablet Weight | | 258.75 | 517.5 | 517.5 |

### Example 2

TABLE 2
Typical Batch Formulae for Controlled Release Carvedilol
Formulations

| Strength | | 50 mg | 50 mg | 50 mg |
|---|---|---|---|---|
| Formula | | BC | BD | BE |
| Component | Compendia | Quantity mg/tablet | | |
| Carvedilol | NC | 1.36 | 0.68 | 0.68 |
| Mannitol | USP | 4.14 | 4.96 | 4.87 |
| Hydroxypropyl Methy- | USP | 1.01 | 1.01 | 1.01 |
| cellulose | | | | |
| Carbomer 934P | NF | 0.20 | 0.05 | 0.14 |
| Magnesium Stearate | NF or Ph. Eur. | 0.07 | 0.07 | 0.07 |
| Opadry White | NC | 0.20 | 0.20 | 0.20 |
| (OY-S-9603) | | | | |
| Opadry Clear | NC | 0.03 | 0.03 | 0.03 |
| (YS-1-19025A) | | | | |
| Purified Water | USP or Ph. Eur. | q.s. | q.s. | q.s. |
| Total Batch Weight (kg) | | 7.0 | 7.0 | 7.0 |
| Batch Size (approx. | | 28,000 | 14,000 | 14,000 |
| number of tablets) | | | | |

### Example 3 (pH Sensitive Coat on Immediate Release Core)

| | % w/w |
|---|---|
| Tablet Core | |
| Carvedilol | 11.45 |
| Lactose | 64.05 |
| Microcrystalline Cellulose | 20.0 |
| Sodium Starch Glycollate | 4.0 |
| Magnesium Stearate | 0.5 |
| TOTAL | 100.0 |
| Tablet Coating (apply approximately 6-10% of tablet core | |
| core weight) | |
| Hydroxypropylmethylcellulose Phthalate | 90.0 |
| Triacetin | 10.0 |
| Example 4 (pH Sensitive Coat on Immediate Release Core) | |
| Tablet Core as in Example 3 | |
| Tablet Coating (apply approximately 6-10% of tablet core | |
| weight) | |
| Cellulose Acetate Phthalate | 90.0 |
| Diethyl Phthalate | 10.0 |
| Example 5 (Controlled Release Coating on Immediate Release | |
| Core) | |
| Tablet Core as in Example 3 | |
| Tablet Coating (apply approximately 5-12% of tablet core | |
| weight) | |
| Eudragit RS 100 | 86.0 |
| Dibutyl Phthalate | 10.0 |
| Talc | 4.0 |
| FD&C Yellow No. 6 | 0.01 |
| Example 6 (pH Sensitive Coat on Controlled Release Core) | |
| Tablet Core as in Example 3 | |
| Tablet Coating as in Example 3 | |
| Example 7 (Encapsulated Controlled Release Coated Beads) | |
| Pellet | (approx) |
| Non Pareil Seed | 30 |
| Carvedilol | 40 |
| Gelatin | 8 |
| Lactose | 20 |
| Talc | 2 |
| Coating | |
| Glycerylmonostearate | 36.6 |
| Glyceryldistearate | 53.4 |
| White Wax | 10.0 |

The foregoing are illustrative of this invention. This invention, however, is not limited to the precise embodiments described herein, but encompasses all modifications within the scope of the claims which follow.

The various references to journals, patents, and other publications which are cited herein comprise the state of the art and are incorporated herein by reference as though fully set forth.

What is claimed is:

1. A matrix formulation comprising carvedilol in an oral dosage unit form.

2. The formulation of claim 1 which comprises a hydrophilic polymer.

3. The formulation of claim 2 wherein the hydrophilic polymer is hydroxypropylmethylcellulose (HMPC).

4. The formulation of claim 1 which comprises a mixture of HMPC and Carbopol.

5. The formulation of claim 1 which comprises a mixture of HMPC, Carbopol and mannitol.

6. An enteric coated formulation comprising carvedilol in an oral dosage unit form.

7. A method of treating hypertension, angina, or congestive heart failure which comprises administering carvedilol in a matrix formulation.

8. A method of treating hypertension, angina, or congestive heart failure which comprises administering carvedilol in an enteric coated formulation.
